(19) United States
(12) Patent Application Publication
(10) Pub. No.: US 2018/0211726 A1
(43) Pub. Date: Jul. 26, 2018

(54) Patient Communication Priority By Compliance Dates, Risk Scores, and Organizational Goals

(71) Applicant: International Business Machines Corporation, Armonk, NY (US)

(72) Inventors: Robert T. Courtemanche, Dallas, TX (US); James S. Cox, Plano, TX (US); Jacob R. Danks, Irving, TX (US); David J. Gattis, Flower Mound, TX (US); Jonathan M. Harmon, Coppell, TX (US); Atul Kumar, Irving, TX (US); Guy B. Mrnustik, Irving, TX (US); Russell G. Olsen, Flower Mound, TX (US)

(21) Appl. No.: 15/828,767

(22) Filed: Dec. 1, 2017

Related U.S. Application Data
(63) Continuation of application No. 15/414,825, filed on Jan. 25, 2017.

(51) Int. Cl. G16H 50/30; G16H 50/20

(52) U.S. Cl. CPC G16H 50/30; G16H 50/20

(57) ABSTRACT

A mechanism is provided in a data processing system comprising least one processor and at least one memory, the at least one memory comprising instructions executed by the at least one processor to cause the at least one processor to implement a clinical decision support system. A priority calculation component executing within the clinical decision support system receives weighted priority configuration for a health care facility and a set of patients with care gaps. The priority calculation component assigns a priority value to each given patient in the set of patients based on a risk score of at least one condition for the given patient, a number of days out of compliance for the at least one condition for the given patient, and an organizational goal factor associated with the at least one condition for the health care organization. The priority calculation component generates a prioritized communication sequence for the set of patients. A communication engine executing within the clinical decision support system initiates communication with patients within the set of patients based on the prioritized communication sequence.

### BACKGROUND

The present application relates generally to an improved data processing apparatus and method and more specifically to mechanisms for patient communication priority by compliance dates, risk scores, and organizational goals.

Decision-support systems exist in many different industries where human experts require assistance in retrieving and analyzing information. An example that will be used throughout this application is a diagnosis system employed in the healthcare industry. Diagnosis systems can be classified into systems that use structured knowledge, systems that use unstructured knowledge, and systems that use clinical decision formulas, rules, trees, or algorithms. The earliest diagnosis systems used structured knowledge or classical, manually constructed knowledge bases. The Internist-I system developed in the 1970's uses disease-finding relations and disease-disease relations. The MYCIN system for diagnosing infectious diseases, also developed in the 1970's, uses structured knowledge in the form of production rules, stating that if certain facts are true, then one can conclude certain other facts with a given certainty factor. DXplain, developed starting in the 1980's, uses structured knowledge similar to that of Internist-I, but adds a hierarchical lexicon of findings.

Iliad, developed starting in the 1990's, adds more sophisticated probabilistic reasoning where each disease has an associated a priori probability of the disease the population for which Iliad was designed), and a list of findings along with the fraction of patients with the disease who have the finding (sensitivity), and the fraction of patients without the disease who have the finding (1-specificity).

In 2000, diagnosis systems using unstructured knowledge started to appear. These systems use some structuring of knowledge such as, for example, entities such as findings and disorders being tagged in documents to facilitate retrieval. ISABEL, for example, uses Autonomy information retrieval software and a database of medical textbooks to retrieve appropriate diagnoses given input findings. Autonomy Auminence uses the Autonomy technology to retrieve diagnoses given findings and organizes the diagnoses by body system. First CONSULT allows one to search a large collection of medical books, journals, and guidelines by chief complaints and age group to arrive at possible diagnoses. PEPID DDX is a diagnosis generator based on PEPID's independent clinical content.

Clinical decision rules have been developed for a number of medical disorders, and computer systems have been developed to help practitioners and patients apply these rules. The Acute Cardiac Ischemia Time-Insensitive Predictive Instrument (ACI-TIPI) takes clinical and ECG features as input and produces probability of acute cardiac ischemia as output to assist with triage of patients with chest pain or other symptoms suggestive of acute cardiac ischemia. ACI-TIPI is incorporated into many commercial heart monitors/defibrillators. The CaseWalker system uses a four-item questionnaire to diagnose major depressive disorder. The PKC Advisor provides guidance on 98 patient problems such as abdominal pain and vomiting.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided in a data processing system comprising at least one processor and at least one memory, the at least one memory comprising instructions executed by the at least one processor to cause the at least one processor to implement a clinical decision support system. The method comprises receiving, by a priority calculation component executing within the clinical decision support system, weighted priority configuration for a health care facility and a set of patients with care gaps. The method further comprises assigning, by the priority calculation component, a priority value to each given patient in the set of patients based on a risk score of at least one condition for the given patient, a number of days out of compliance for the at least one condition for the given patient, and an organizational goal factor associated with the at least one condition for the health care organization. The method further comprises generating, by the priority calculation component, a prioritized communication sequence for the set of patients. The method further comprises initiating, by a communication engine executing within the clinical decision support system, communication with patients within the set of patients based on the prioritized communication sequence.

In other illustrative embodiments, a computer program product comprising a computer usable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 depicts a schematic diagram of one illustrative embodiment of a cognitive system in a computer network;

FIG. 2 is a block diagram of an example data processing system in which aspects of the illustrative embodiments are implemented;

FIG. 3 is an example diagram illustrating an interaction of elements of a healthcare cognitive system in accordance with one illustrative embodiment;

FIG. 4 is a block diagram of a patient communication priority mechanism in accordance with an illustrative embodiment;

FIG. 5 depicts a weighted priority configuration for patient communication priority in accordance with an illustrative embodiment;

FIGS. 6A-6C depict example patient information for patients with care gaps in accordance with an illustrative embodiment; and

FIG. 7 is a flowchart illustrating operation of a system for patient communication priority by compliance dates, risk scores, and organizational goals in accordance with an illustrative embodiment.

### DETAILED DESCRIPTION

Medical organizations, physicians, and care managers attempt to close care gaps to improve patient health and to meet goals across large patient populations. Population health management systems provide mechanisms to automatically generate communications to patients to close care gaps. This meets three primary goals: revenue generation for the medical facility, improved performance metrics, and, most importantly, improve patient outcomes.

One problem with this solution is prioritization of communications. Dealing with large patient populations, calling all patients with care gaps is not effective, because that would generate too much activity for a facility with limited capacity to handle. One solution is to throttle communications by limiting the number of calls per day based on expected capacity. The priorities of the calls may be based on a fixed priority list based on the underlying condition associated with the targeted communication. However, a fixed priority may not align with the overall goals of the organization. Furthermore, a fixed priority may not prioritize patients who are beyond their specific care gaps.

The illustrative embodiments provide a mechanism to assist with selection of provider or facility for surgical procedures based on frequency of the procedure, history of complication, and cost. The mechanism provides a comprehensive screening and search interface that provides data analysis and selection tools that allow users to screen available providers and facilities for specific procedures. The mechanism allows care managers, clinical staff, and patients to review available providers and facilities within a selectable geographic region. The mechanism allows the user to screen, view, and filter providers and facilities displayed based on risk of complications for specific procedures. The mechanism also provides an overlay of cost estimates and ranges, including total cost for the procedure, cost to the insurer, and out-of-pocket costs to the patient, along with in-network and out-of-network breakdowns based on the patient's health insurance plan.

Before beginning the discussion of the various aspects of the illustrative embodiments in more detail, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general purpose hardware, a procedure or method for executing the functions, or a combination of any of the above.

The present description and claims may make use of the terms “a,” “at least one of,” and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “component,” if used herein with regard to describing embodiments and features of the invention, is not intended to be limiting of any particular implementation for accomplishing and/or performing the actions, steps, processes, etc., attributable to and/or performed by the component. A component may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular component is, unless otherwise specified, for purposes of convenience of reference and not intended to be limiting to a specific implementation. Additionally, any functionality attributed to a component may be equally performed by multiple components, incorporated into and/or combined with the functionality of another component of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

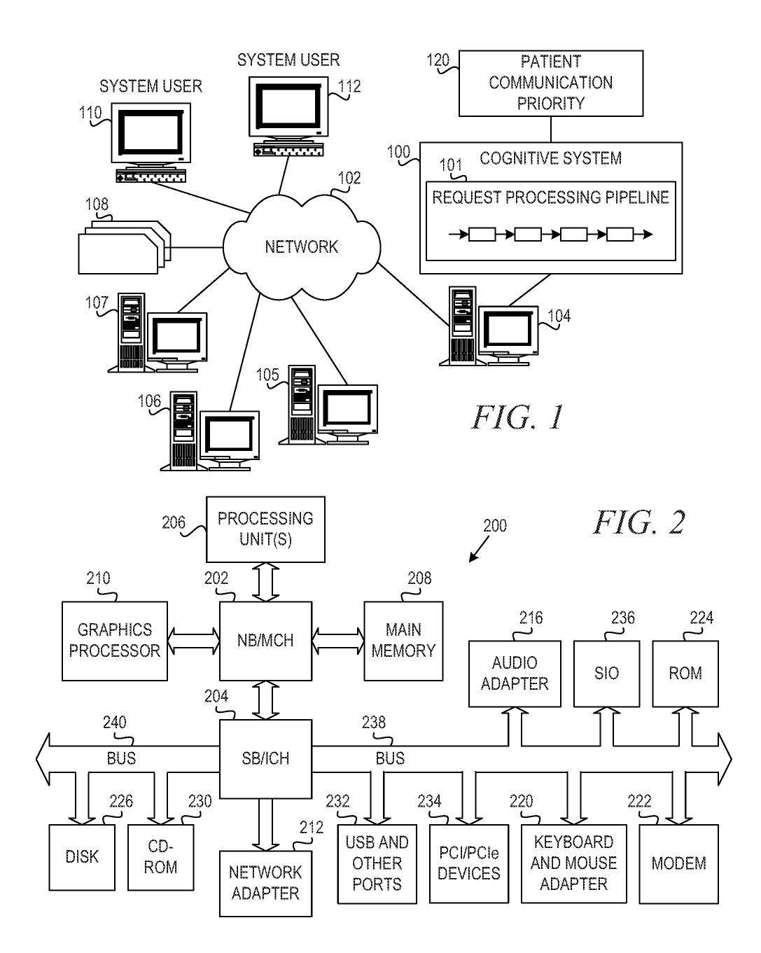

The illustrative embodiments may be utilized in many different types of data processing environments. In order to provide a context for the description of the specific elements and functionality of the illustrative embodiments, FIGS. 1-3 are provided hereafter as example environments in which aspects of the illustrative embodiments may be implemented. It should be appreciated that FIGS. 1-3 are only examples and are not intended to assert or imply any limitation with regard to the environments in which aspects or embodiments of the present invention may be implemented. Many modifications to the depicted environments may be made without departing from the spirit and scope of the present invention.

FIGS. 1-3 are directed to describing an example cognitive system for healthcare applications (also referred to herein as a “healthcare cognitive system”) which implements a request processing pipeline, such as a Question Answering (QA) pipeline (also referred to as a Question/Answer pipeline or Question and Answer pipeline) for example, request processing methodology, and request processing computer program product with which the mechanisms of the illustrative embodiments are implemented. These requests may be provided as structured or unstructured request messages, natural language questions, or any other suitable format for requesting an operation to be performed by the healthcare cognitive system. As described in more detail hereafter, the particular healthcare application that is implemented in the cognitive system of the present invention is a healthcare application for providing medical treatment recommendations for patients based on their specific features as obtained from various sources, e.g., patient electronic medical records (EMRs), patient questionnaires, etc. In particular, the mechanisms of the present invention provide a mechanism for patient communication prioritization by compliance dates, risk scores, and organizational goals.

It should be appreciated that the healthcare cognitive system, while shown as having a single request processing pipeline in the examples hereafter, may in fact have multiple request processing pipelines. Each request processing pipeline may be separately trained and/or configured to process requests associated with different domains or be configured to perform the same or different analysis on input requests, depending on the desired implementation. For example, in some cases, a first request processing pipeline may be trained to operate on input requests directed to a first medical malady domain (e.g., various types of blood diseases) while another request processing pipeline may be trained to answer input requests in another medical malady domain (e.g., various types of cancers). In other cases, for example, the request processing pipelines may be configured to provide different types of cognitive functions or support different types of healthcare applications, such as one request processing pipeline being used for patient diagnosis, another request processing pipeline being configured for medical treatment recommendation, another request processing pipeline being configured for patient monitoring, etc.

Moreover, each request processing pipeline may have its own associated corpus or corpora that it ingests and operates on, e.g., one corpus for blood disease domain documents and another corpus for cancer diagnostics domain related documents in the above examples. In some cases, the request processing pipelines may each operate on the same domain of input questions but may have different configurations, e.g., different annotators or differently trained annotators, such that different analysis and potential answers are generated. The healthcare cognitive system may provide additional logic for routing input requests to the appropriate request processing pipeline, such as based on a determined domain of the input request, combining and evaluating final results generated by the processing performed by multiple request processing pipelines, and other control and interaction logic that facilitates the utilization of multiple request processing pipelines.

As an overview, a cognitive system is a specialized computer system, or set of computer systems, configured with hardware and/or software logic (in combination with hardware logic upon which the software executes) to emulate human cognitive functions. These cognitive systems apply human-like characteristics to conveying and manipulating ideas which, when combined with the inherent strengths of digital computing, can solve problems with high accuracy and resilience on a large scale. A cognitive system performs one or more computer-implemented cognitive operations that approximate a human thought process as well as enable people and machines to interact in a more natural manner so as to extend and magnify human expertise and cognition. A cognitive system comprises artificial intelligence logic, such as natural language processing (NLP) based logic, for example, and machine learning logic, which may be provided as specialized hardware, software executed on hardware, or any combination of specialized hardware and software executed on hardware. The logic of the cognitive system implements the cognitive operation(s), examples of which include, but are not limited to, question answering, identification of related concepts within different portions of content in a corpus, intelligent search algorithms, such as Internet web page searches, for example, medical diagnostic and treatment recommendations, and other types of recommendation generation, e.g., items of interest to a particular user, potential new contact recommendations, or the like.

IBM Watson™ is an example of one such cognitive system which can process human readable language and identify inferences between text passages with human-like high accuracy at speeds far faster than human beings and on a larger scale. In general, such cognitive systems are able to perform the following functions:
Navigate the complexities of human language and understanding
Ingest and process vast amounts of structured and unstructured data
Generate and evaluate hypothesis
Weigh and evaluate responses that are based only on relevant evidence
Provide situation-specific advice, insights, and guidance
Improve knowledge and learn with each iteration and interaction through machine learning processes
Enable decision making at the point of impact (contextual guidance)
Scale in proportion to the task
Extend and magnify human expertise and cognition
Identify resonating, human-like attributes and traits from natural language
Deduce various language specific or agnostic attributes from natural language
High degree of relevant recollection from data points (images, text, voice) (memorization and recall)
Predict and sense with situational awareness that mimic human cognition based on experiences
Answer questions based on natural language and specific evidence

In one illustrative embodiment, the cognitive system may use existing cognitive tools or services, such as the IBM Watson™ Services provided through the IBM Watson™ Developer Cloud. The cognitive system may use application programming interfaces (APIs) to access these cognitive tools or services. As an example, the cognitive system may use a statistical analysis tool or service, such as the IBM Watson™ Tradeoff Analytics service available through the IBM Watson™ Services provided through the IBM Watson™ Developer Cloud. The IBM Watson™ Tradeoff Analytics service helps people make better choices when faced with multiple, often conflicting goals and alternatives. By using mathematical filtering techniques to identify the top options based on multiple criteria, the service can help decision makers explore the trade-offs between options when making complex decisions. The service combines smart visualization and analytical recommendations for easy and intuitive exploration of trade-offs. A user specifies objectives, preferences, and priorities; the service filters out less attractive options to encourage the user's exploration of the remaining optimal candidates. In this way, the service helps decision makers consider only the goals that matter most and only the best options to make a final, informed decision.

FIG. 1 depicts a schematic diagram of one illustrative embodiment of a cognitive system 100 implementing a request processing pipeline 101, which in some embodiments may be a question answering (QA) pipeline, in a computer network 102. For purposes of the present description, it will be assumed that the request processing pipeline 101 is implemented as a QA pipeline that operates on structured and/or unstructured requests in the form of input questions. One example of a question processing operation which may be used in conjunction with the principles described herein is described in U.S. Patent Application Publication No. 2011/0125734, which is herein incorporated by reference in its entirety.

The cognitive system 100 is implemented on one or more computing devices 104-107 (comprising one or more processors and one or more memories, and potentially any other computing device elements generally known in the art including buses, storage devices, communication interfaces, and the like) connected to the computer network 102. The network 102 includes multiple computing devices 104-107 in communication with each other and with other devices or components via one or more wired and/or wireless data communication links, where each communication link comprises one or more of wires, routers, switches, transmitters, receivers, or the like. The cognitive system 100 and network 102 enables question processing and answer generation (QA) functionality for one or more cognitive system users via their respective computing devices 110, 112. Other embodiments of the cognitive system 100 may be used with components, systems, sub-systems, and/or devices other than those that are depicted herein.

The cognitive system 100 is configured to implement a request processing pipeline 101 that receive inputs from various sources. For example, the cognitive system 100 receives input from the network 102, a corpus of electronic documents 108, cognitive system users, and/or other data and other possible sources of input. In one embodiment, some or all of the inputs to the cognitive system 100 are routed through the network 102. The various computing devices 104 on the network 102 include access points for content creators and QA system users. Some of the computing devices 104-107 include devices for a database storing the corpus of data 108 (which is shown as a separate entity in FIG. 1 for illustrative purposes only). Portions of the corpus of data 108 may also be provided on one or more other network attached storage devices, in one or more databases, or other computing devices not explicitly shown in FIG. 1. The network 102 includes local network connections and remote connections in various embodiments, such that, the cognitive system 100 may operate in environments of any size, including local and global, e.g., the Internet.

In one embodiment, the content creator creates content in a document of the corpus of data 108 for use as part of a corpus of data with the cognitive system 100. The document includes any file, text, article, or source of data for use in the cognitive system 100. Cognitive system users access the cognitive system 100 via a network connection or an Internet connection to the network 102, and input questions to the cognitive system 100 that are answered by the content in the corpus of data 108. In one embodiment, the questions are formed using natural language. The cognitive system 100 parses and interprets the question via a request processing pipeline 101, and provides a response to the cognitive system user, e.g., cognitive system user 110, containing one or more answers to the question. In some embodiments, the cognitive system 100 provides a response to users in a ranked list of candidate answers while in other illustrative embodiments, the cognitive system 100 provides a single final answer or a combination of a final answer and ranked listing of other candidate answers.

The cognitive system 100 implements the request processing pipeline 101, which comprises a plurality of stages for processing an input question and the corpus of data 108. The request processing pipeline 101 generates answers for the input question based on the processing of the input question and the corpus of data 108.

In some illustrative embodiments, the cognitive system 100 may be the IBM Watson™ cognitive system available from International Business Machines Corporation of Armonk, N.Y., which is augmented with the mechanisms of the illustrative embodiments described hereafter. As outlined previously, a request processing pipeline of the IBM Watson™ cognitive system receives an input question which it then parses to extract the major features of the question, which in turn are then used to formulate queries that are applied to the corpus of data. Based on the application of the queries to the corpus of data, a set of hypotheses, or candidate answers to the input question, are generated by looking across the corpus of data for portions of the corpus of data that have some potential for containing a valuable response to the input question. The request processing pipeline of the IBM Watson™ cognitive system then performs deep analysis on the language of the input question and the language used in each of the portions of the corpus of data found during the application of the queries using a variety of reasoning algorithms.

The scores obtained from the various reasoning algorithms are then weighted against a statistical model that summarizes a level of confidence that the request processing pipeline of the IBM Watson™ cognitive system has regarding the evidence that the potential response, i.e. candidate answer, is inferred by the question. This process is be repeated for each of the candidate answers to generate ranked listing of candidate answers which may then be presented to the user that submitted the input question, or from which a final answer is selected and presented to the user. More information about the request processing pipeline of the IBM Watson™ cognitive system may be obtained, for example, from the IBM Corporation website, IBM Redbooks, and the like. For example, information about the request processing pipeline of the IBM Watson™ cognitive system can be found in Yuan et al., “Watson and Healthcare,” IBM developerWorks, 2011 and “The Era of Cognitive Systems: An Inside Look at IBM Watson and How it Works” by Rob High, IBM Redbooks, 2012.

As noted above, while the input to the cognitive system 100 from a client device may be posed in the form of a natural language question, the illustrative embodiments are not limited to such. Rather, the input question may in fact be formatted or structured as any suitable type of request which may be parsed and analyzed using structured and/or unstructured input analysis, including but not limited to the natural language parsing and analysis mechanisms of a cognitive system such as the IBM Watson™ cognitive system, to determine the basis upon which to perform cognitive analysis and providing a result of the cognitive analysis. In the case of a healthcare based cognitive system, this analysis may involve processing patient medical records, medical guidance documentation from one or more corpora, and the like, to provide a healthcare oriented cognitive system result.

In the context of the present invention, cognitive system 100 may provide a cognitive functionality for assisting with healthcare based operations. For example, depending upon the particular implementation, the healthcare based operations may comprise patient diagnostics, medical treatment recommendation systems, medical practice management systems, personal patient care plan generation and monitoring, patient electronic medical record (EMR) evaluation for various purposes, such as for identifying patients who have care gaps, patients who are high risk, or the like. Thus, the cognitive system 100 may be a healthcare decision support system that operates in the medical or healthcare type domains and which may process requests for such healthcare operations via the request processing pipeline 106 input as either structured or unstructured requests, natural language input questions, or the like. In one illustrative embodiment, the cognitive system 100 is a medical treatment recommendation system that analyzes a patient's EMR in relation to medical guidelines and other medical documentation in a corpus of information to generate a recommendation as to how to treat a medical malady or medical condition of the patient. A patient's EMR may contain structured and unstructured information that comes from an Electronic Health Record (UR) system, which may further be augmented with information from a clinician when using a clinical decision support system.

In particular, the cognitive system 100 implements a patient communication priority component 120 for patient communication priority by compliance dates, risk scores, and organizational goals. While prioritizing communications based on the underlying condition is a good solution, it does not go far enough. The illustrative embodiments take the concept further so that patients with higher risk scores associated with the condition and who are further out of compliance are prioritized over those who have lower risk scores and who are closer to being in compliance, combined with the seriousness of the condition. A second factor taken into account by the illustrative embodiments is the goal of the organization, targeted metrics for the organization or physician, to drive more patients to goal for those metrics. Communication priority is a factor of those two metrics, balancing high risk patient needs with organizational goals.

FIG. 2 is a block diagram of an example data processing system in which aspects of the illustrative embodiments are implemented. Data processing system 200 is an example of a computer, such as server 104 or client 110 in FIG. 1, in which computer usable code or instructions implementing the processes for illustrative embodiments of the present invention are located. In one illustrative embodiment, FIG. 2 represents a server computing device, such as a server 104, which implements a cognitive system 100 and cognitive system pipeline 101 augmented to include the additional mechanisms of the illustrative embodiments described hereafter.

In the depicted example, data processing system 200 employs a hub architecture including north bridge and memory controller hub (NB/MCH) 202 and south bridge and input/output (I/O) controller hub (SB/ICH) 204. Processing unit 206, main memory 208, and graphics processor 210 are connected to NB/MCH 202. Graphics processor 210 is connected to NB/MCH 202 through an accelerated graphics port (AGP).

In the depicted example, local area network (LAN) adapter 212 connects to SB/ICH 204. Audio adapter 216, keyboard and mouse adapter 220, modem 222, read only memory (ROM) 224, hard disk drive (HDD) 226, CD-ROM drive 230, universal serial bus (USB) ports and other communication ports 232, and PCI/PCIe devices 234 connect to SB/ICH 204 through bus 238 and bus 240. PCI/PCIe devices may include, for example, Ethernet adapters, add-in cards, and PC cards for notebook computers. PCI uses a card bus controller, while PCIe does not. ROM 224 may be, for example, a flash basic input/output system (BIOS).

HDD 226 and CD-ROM drive 230 connect to SB/ICH 204 through bus 240, HDD 226 and CD-ROM drive 230 may use, for example, an integrated drive electronics (IDE) or serial advanced technology attachment (SATA) interface. Super I/O (SIO) device 236 is connected to SB/ICH 204.

An operating system runs on processing unit 206. The operating system coordinates and provides control of various components within the data processing system 200 in FIG. 2. As a client, the operating system is a commercially available operating system such as Microsoft® Windows 8®. An object-oriented programming system, such as the Java™ programming system, may run in conjunction with the operating system and provides calls to the operating system from Java™ programs or applications executing on data processing system 200.

As a server, data processing system 200 may be, for example, an IBM® eServer™ System p® computer system, running the Advanced Interactive Executive (AIX®) operating system or the LINUX® operating system. Data processing system 200 may be a symmetric multiprocessor (SMP) system including a plurality of processors in processing unit 206. Alternatively, a single processor system may be employed.

Instructions for the operating system, the object-oriented programming system, and applications or programs are located on storage devices, such as HDD 226, and are loaded into main memory 208 for execution by processing unit 206. The processes for illustrative embodiments of the present invention are performed by processing unit 206 using computer usable program code, which is located in a memory such as, for example, main memory 208, ROM 224, or in one or more peripheral devices 226 and 230, for example.

A bus system, such as bus 238 or bus 240 as shown in FIG. 2, is comprised of one or more buses. Of course, the bus system may be implemented using any type of communication fabric or architecture that provides for a transfer of data between different components or devices attached to the fabric or architecture. A communication unit, such as modem 222 or network adapter 212 of FIG. 2, includes one or more devices used to transmit and receive data. A memory may be, for example, main memory 208, ROM 224, or a cache such as found in NB/MCH 202 in FIG. 2.

Those of ordinary skill in the art will appreciate that the hardware depicted in FIGS. 1 and 2 may vary depending on the implementation. Other internal hardware or peripheral devices, such as flash memory, equivalent non-volatile memory, or optical disk drives and the like, may be used in addition to or in place of the hardware depicted in FIGS. 1 and 2. Also, the processes of the illustrative embodiments may be applied to a multiprocessor data processing system, other than the SMP system mentioned previously, without departing from the spirit and scope of the present invention.

Moreover, the data processing system 200 may take the form of any of a number of different data processing systems including client computing devices, server computing devices, a tablet computer, laptop computer, telephone or other communication device, a personal digital assistant (PDA), or the like. In some illustrative examples, data processing system 200 may be a portable computing device that is configured with flash memory to provide non-volatile memory for storing operating system files and/or user-generated data, for example. Essentially, data processing system 200 may be any known or later developed data processing system without architectural limitation.

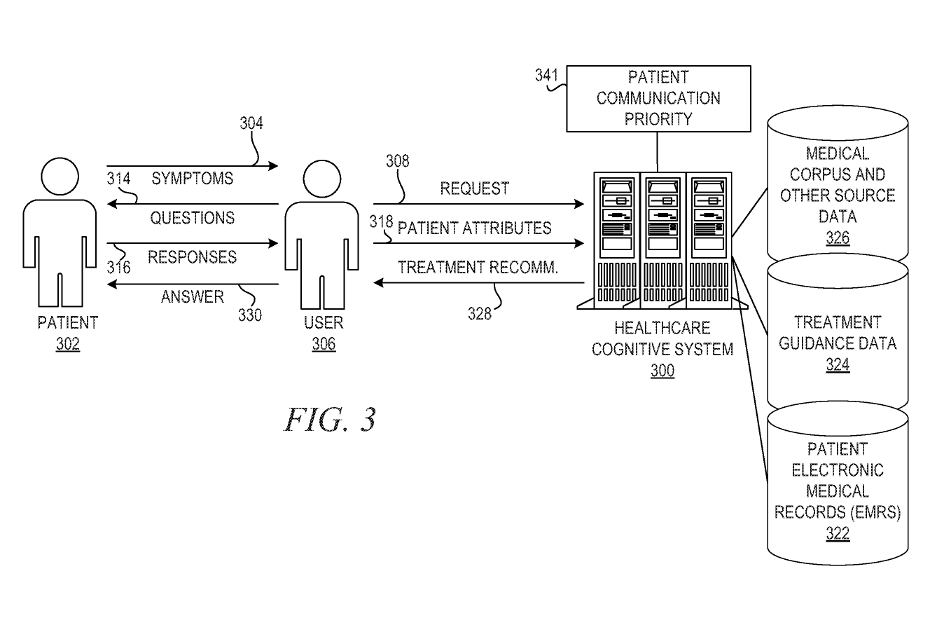

FIG. 3 is an example diagram illustrating an interaction of elements of a healthcare cognitive system in accordance with one illustrative embodiment. The example diagram of FIG. 3 depicts an implementation of a healthcare cognitive system 300 that is configured to provide medical treatment recommendations for patients. However, it should be appreciated that this is only an example implementation and other healthcare operations may be implemented in other embodiments of the healthcare cognitive system 300 without departing from the spirit and scope of the present invention.

Moreover, it should be appreciated that while FIG. 3 depicts the patient 302 and user 306 as human figures, the interactions with and between these entities may be performed using computing devices, medical equipment, and/or the like, such that entities 302 and 306 may in fact be computing devices, e.g., client computing devices. For example, the interactions 304, 314, 316, and 330 between the patient 302 and the user 306 may be performed orally, e.g., a doctor interviewing a patient, and may involve the use of one or more medical instruments, monitoring devices, or the like, to collect information that may be input to the healthcare cognitive system 300 as patient attributes 318. Interactions between the user 306 and the healthcare cognitive system 300 will be electronic via a user computing device (not shown), such as a client computing device 110 or 112 in FIG. 1, communicating with the healthcare cognitive system 300 via one or more data communication links and potentially one or more data networks.

As shown in FIG. 3, in accordance with one illustrative embodiment, a patient 302 presents symptoms 304 of a medical malady or condition to a user 306, such as a healthcare practitioner, technician, or the like. The user 306 may interact with the patient 302 via a question 314 and response 316 exchange where the user gathers more information about the patient 302, the symptoms 304, and the medical malady or condition of the patient 302. It should be appreciated that the questions/responses may in fact also represent the user 306 gathering information from the patient 302 using various medical equipment, e.g., blood pressure monitors, thermometers, wearable health and activity monitoring devices associated with the patient such as a FitBit™ wearable device, a wearable heart monitor, or any other medical equipment that may monitor one or more medical characteristics of the patient 302. In some cases such medical equipment may be medical equipment typically used in hospitals or medical centers to monitor vital signs and medical conditions of patients that are present in hospital beds for observation or medical treatment.

In response, the user 302 submits a request 308 to the healthcare cognitive system 300, such as via a user interface on a client computing device that is configured to allow users to submit requests to the healthcare cognitive system 300 in a format that the healthcare cognitive system 300 can parse and process. The request 308 may include, or be accompanied with, information identifying patient attributes 318. These patient attributes 318 may include, for example, an identifier of the patient 302 from which patient EMRs 322 for the patient may be retrieved, demographic information about the patient, the symptoms 304, and other pertinent information obtained from the responses 316 to the questions 314 or information obtained from medical equipment used to monitor or gather data about the condition of the patient 302. Any information about the patient 302 that may be relevant to a cognitive evaluation of the patient by the healthcare cognitive system 300 may be included in the request 308 and/or patient attributes 318.

The healthcare cognitive system 300 provides a cognitive system that is specifically configured to perform an implementation specific healthcare oriented cognitive operation. In the depicted example, this healthcare oriented cognitive operation is directed to providing a treatment recommendation 328 to the user 306 to assist the user 306 in treating the patient 302 based on their reported symptoms 304 and other information gathered about the patient 302 via the question 314 and response 316 process and/or medical equipment monitoring/data gathering. The healthcare cognitive system 300 operates on the request 308 and patient attributes 318 utilizing information gathered from the medical corpus and other source data 326, treatment guidance data 324, and the patient EMRs 322 associated with the patient 302 to generate one or more treatment recommendation 328. The treatment recommendations 328 may be presented in a ranked ordering with associated supporting evidence, obtained from the patient attributes 318 and data sources 322-326, indicating the reasoning as to why the treatment recommendation 328 is being provided and why it is ranked in the manner that it is ranked.

For example, based on the request 308 and the patient attributes 318, the healthcare cognitive system 300 may operate on the request, such as by using a request processing pipeline type processing, to parse the request 308 and patient attributes 318 to determine what is being requested and the criteria upon which the request is to be generated as identified by the patient attributes 318, and may perform various operations for generating queries that are sent to the data sources 322-326 to retrieve data, generate candidate treatment recommendations (or answers to the input question), and score these candidate treatment recommendations based on supporting evidence found in the data sources 322-326. In the depicted example, the patient EMRs 322 is a patient information repository that collects patient data from a variety of sources, e.g., hospitals, laboratories, physicians' offices, health insurance companies, pharmacies, etc. The patient EMRs 322 store various information about individual patients, such as patient 302, in a manner (structured, unstructured, or a mix of structured and unstructured formats) that the information may be retrieved and processed by the healthcare cognitive system 300. This patient information may comprise varied demographic information about patients, personal contact information about patients, employment information, health insurance information, laboratory reports, physician reports from office visits, hospital charts, historical information regarding previous diagnoses, symptoms, treatments, prescription information, etc. Based on an identifier of the patient 302, the patient's corresponding EMRs 322 from this patient repository may be retrieved by the healthcare cognitive system 300 and searched/processed to generate treatment recommendations 328.

The treatment guidance data 324 provides a knowledge base of medical knowledge that is used to identify potential treatments tor a patient based on the patient's attributes 318 and historical information presented in the patient's EMRs 322. This treatment guidance data 324 may be obtained from official treatment guidelines and policies issued by medical authorities, e.g., the American Medical Association, may be obtained from widely accepted physician medical and reference texts, e.g., the Physician's Desk Reference, insurance company guidelines, or the like. The treatment guidance data 324 may be provided in any suitable form that may be ingested by the healthcare cognitive system 300 including both structured and unstructured formats. In one embodiment, the treatment guidance data 324 may include weighted priority configuration information, such as compliance priorities by reason, risk priority factors, and organizational goal factors.

In some cases, such treatment guidance data 324 may be provided in the form of rules that indicate the criteria required to be present, and/or required not to be present, tor the corresponding treatment to be applicable to a particular patient for treating a particular symptom or medical malady/condition. For example, the treatment guidance data 324 may comprise a treatment recommendation rule that indicates that for a treatment of Decitabine, strict criteria for the use of such a treatment is that the patient 302 is less than or equal to 60 years of age, has acute myeloid leukemia (AML), and no evidence of cardiac disease. Thus, for a patient 302 that is 59 years of age, has AML, and does not have any evidence in their patient attributes 318 or patient EMRs indicating evidence of cardiac disease, the following conditions of the treatment rule exist:
Age<=60 years=59 (MET);
Patient has AML=AML (MET); and
Cardiac Disease=false (MET)
   Since all of the criteria of the treatment rule are met by the specific information about this patient 302, then the treatment of Decitabine is a candidate treatment for consideration for this patient 302. However, if the patient had been 69 years old, the first criterion would not have been met and the Decitabine treatment would not be a candidate treatment for consideration for this patient 302. Various potential treatment recommendations may be evaluated by the healthcare cognitive system 300 based on ingested treatment guidance data 324 to identify subsets of candidate treatments for further consideration by the healthcare cognitive system 300 by scoring such candidate treatments based on evidential data obtained from the patient EMRs 322 and medical corpus and other source data 326.

For example, data mining processes may be employed to mine the data in sources 322 and 326 to identify evidential data supporting and/or refuting the applicability of the candidate treatments to the particular patient 302 as characterized by the patient's patient attributes 318 and EMRs 322. For example, for each of the criteria of the treatment rule, the results of the data mining provides a set of evidence that supports giving the treatment in the cases where the criterion is “MET” and in cases where the criterion is “NOT MET.” The healthcare cognitive system 300 processes the evidence in accordance with various cognitive logic algorithms to generate a confidence score for each candidate treatment recommendation indicating a confidence that the corresponding candidate treatment recommendation is valid for the patient 302. The candidate treatment recommendations may then be ranked according to their confidence scores and presented to the user 306 as a ranked listing of treatment recommendations 328. In some cases, only a highest ranked, or final answer, is returned as the treatment recommendation 328. The treatment recommendation 328 may be presented to the user 306 in a manner that the underlying evidence evaluated by the healthcare cognitive system 300 may be accessible, such as via a drilldown interface, so that the user 306 may identify the reasons why the treatment recommendation 328 is being provided by the healthcare cognitive system 300.

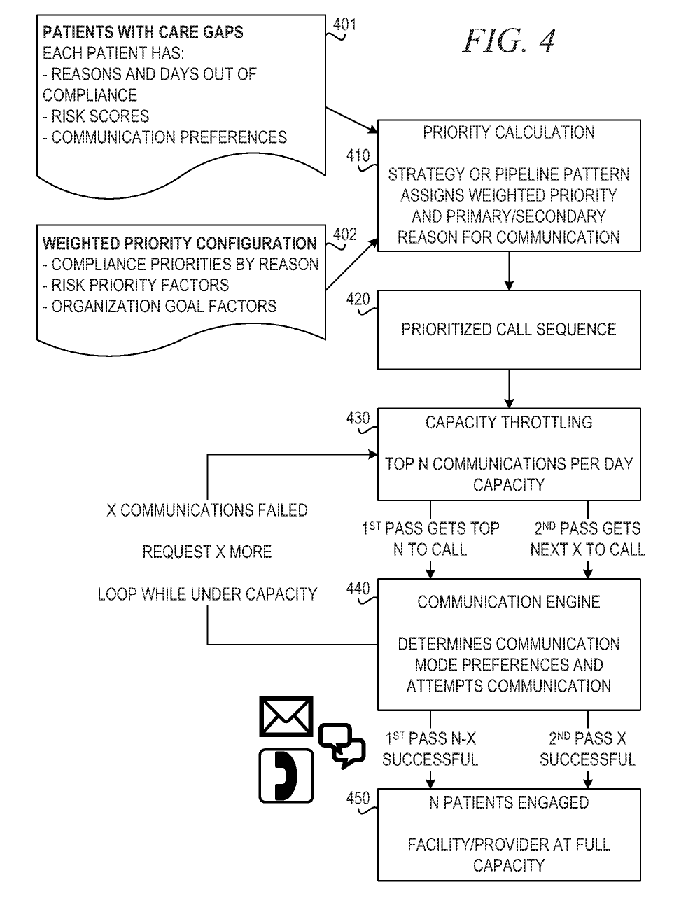

In accordance with the illustrative embodiments herein, the healthcare cognitive system 300 is augmented to operate with, implement, or include patient communication priority component 341 for patient communication priority by compliance dates, risk scores, and organizational goals. While the above description describes a general healthcare cognitive system 300 that may operate on specifically configured treatment recommendation rules, the mechanisms of the illustrative embodiments modify such operations to utilize the patient communication priority component 341, which operates in the manner described with particular reference to FIGS. 4-7 below.

Thus, in response to the healthcare cognitive system 300 receiving the request 308 and patient attributes 318, the healthcare cognitive system 300 may retrieve the patient's EMR clinical history, demographics, biometric data, claims data, genomic data, and health insurance plan data from source(s) 322, 324, 326. This information is provided to patient communication priority component 341, which generates a prioritized call sequence for communicating with patients. Patient communication priority component 341 receives patient information from patient EMRs 322 and assigns a weighted priority based on days out of compliance, risk scores, and communication preferences, as well as risk priority factors and organizational goals of the provider/facility. Patient communication priority component 341 presents the prioritized call sequence to perform human initiated or machine-generated communications. In response to a number of communications not being successful, patient communication priority component 341 repeats the process until the provider/facility is at full capacity.

While FIG. 3 is depicted with an interaction between the patient 302 and a user 306, which may be a healthcare practitioner such as a physician, nurse, physician's assistant, lab technician, or any other healthcare worker, for example, the illustrative embodiments do not require such. Rather, the patient 302 may interact directly with the healthcare cognitive system 300 without having to go through an interaction with the user 306 and the user 306 may interact with the healthcare cognitive system 300 without having to interact with the patient 302. For example, in the first case, the patient 302 may be requesting 308 treatment recommendations 328 from the healthcare cognitive system 300 directly based on the symptoms 304 provided by the patient 302 to the healthcare cognitive system 300. Moreover, the healthcare cognitive system 300 may actually have logic for automatically posing questions 314 to the patient 302 and receiving responses 316 from the patient 302 to assist with data collection for generating treatment recommendations 328. In the latter case, the user 306 may operate based on only information previously gathered and present in the patient EMR 322 by sending a request 308 along with patient attributes 318 and obtaining treatment recommendations in response from the healthcare cognitive system 300. Thus, the depiction in FIG. 3 is only an example and should not be interpreted as requiring the particular interactions depicted when many modifications may be made without departing from the spirit and scope of the present invention.

FIG. 4 is a block diagram of a patient communication priority mechanism in accordance with an illustrative embodiment. Priority calculation component 410 receives as input patient data for patients with care gaps 401 and weighted priority configuration 402. The input data for patients with care gaps 401 may include for each patient: reasons and days out of compliance, risk scores, and communication preference of the patient. For instance, a patient may prefer to be contacted by telephone, text, electronic mail, or any other mode of communication. The days out of compliance may be determined from patient EMRs. The risk scores may be based on historical information.

The weighted priority configuration 402 may include compliance priorities by reason, which could be simple priority weights or may be rules-based stored as a set of rules, risk priority factors, and organization goal factors. The organizational goals may be driven by contractual obligations, insurance incentives, cost, Medicare goals, provider/facility marks that need improvement, etc. In one example embodiment, the illustrative embodiments may provide a user interface (not shown) that allows a user to adjust dials or sliders to balance organizational goals vs. risk factors, high-risk patients vs. preventive treatment, or the like.

In priority calculation component 410, a strategy or pipeline pattern assigns a weighted priority and primary/secondary reason for communication for each patient in the input data for patients with care gaps 401. Priority calculation component 410 generates priority call sequence 420.

Capacity throttling component 430 considers the top N communications per day capacity, where N is determined based on the full capacity of the facility or provider. The full capacity may be the full communication capacity to communicate with patients throughout the day. Alternatively, N may be determined based on an expected number of successful communications that would result in a full day's worth of in-facility appointments, including successful communications that do not result in an appointment or no-shows.

In a first pass, capacity throttling component 430 sends the top N patients to communication engine 440, whish determines the communication mode preferences and attempts communication. In the depicted example, X number of patients are not contacted successfully in the first pass, and communication is successful for N-X patients. Communication engine 440 then requests X more patients, and this process loops until full capacity is reached.

In the depicted example, communication is successful for the X number of patients. Thus, after the second pass, N patients are engaged and in component 450, the facility/provider is at full capacity.

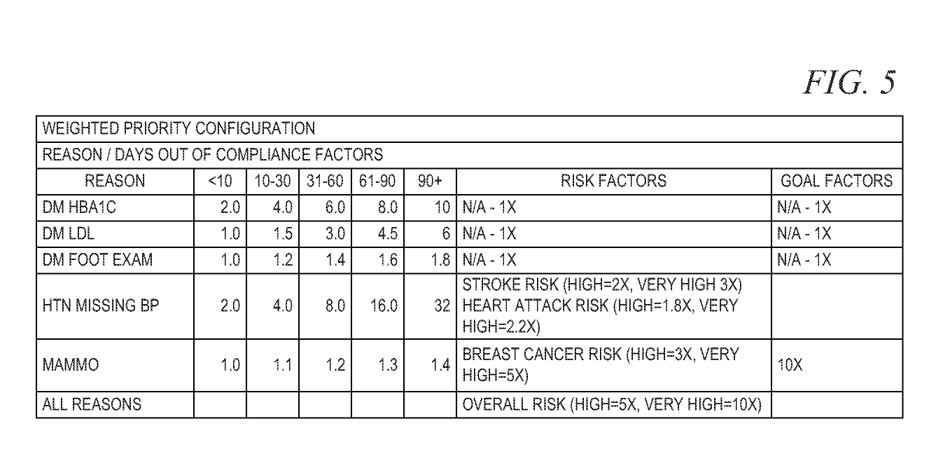

FIG. 5 depicts an example weighted priority configuration for patient communication priority in accordance with an illustrative embodiment. The depicted example shows five different reasons for risk/compliance: DM HBA1C (hemoglobin testing for diabetes), DM LDL (cholesterol testing for diabetes), DM FOOT EXAM (test for diabetes-related foot complications), HTN MISSING BP (hypertension (high blood pressure) testing), and MAMMO (mammogram for breast cancer detection), as well as an ALL REASONS category. The weighted priority configuration includes weights for five categories of days out of compliance: <10, 10-30, 31-60, 61-90, and 90+. The weighted priority configuration shown in FIG. 5 also includes risk factors and goal factors for a given facility or provider.

As shown in the example of FIG. 5, HTN MISSING BP has comparatively high weights for days out of compliance and different risk factors based on stroke risk and heart attack risk. MAMMO also has different risk factors based on breast cancer risk and has a comparatively higher goal factor.

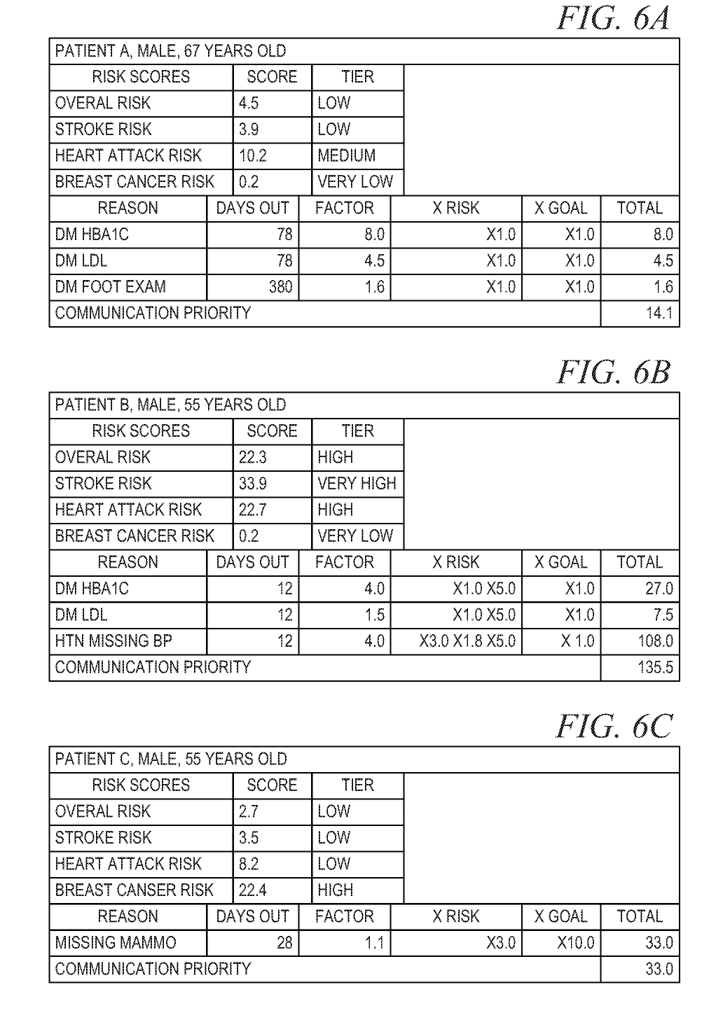

FIGS. 6A-6C depict example patient information for patients with care gaps in accordance with an illustrative embodiment. FIG. 6A shows a patient that is a 67-year-old male with risk scores for overall risk (4.5 low), stroke risk (3.9 low), heart attack risk (10.2 medium), and breast cancer risk (0.2 very low). The patient depicted in FIG. 6A is 78 days out for DM HBA1C with a total risk score of 8.0, 78 days out for DM LDL with a total risk score of 4.5, and 380 days out for DM FOOT EXAM with a total risk score of 1.6. These risk scores result in a communication priority of 14.1. The patient depicted in FIG. 6A is assigned a primary reason for communication of DM HBA1C and a secondary reason of DM LDL based on the respective risk scores.

FIG. 6B shows a patient that is a 55-year-old male with risk scores for overall risk (22.3 high), stroke risk (33.9 very high), heart attack risk (22.7 high), and breast cancer risk (0.2 very low). The patient depicted in FIG. 6B is 12 days out for DM HBA1C with a total risk score of 27.0, 12 days out for DM LDL with a total risk score of 7.5, and 12 days out for HTN MISSING BP with a total risk score of 108.0. These risk scores result in a communication priority of 135.5. Each patient gets an additional risk factor applied for the “Overall Risk” for the patient. This is an overall risk assessment used to gauge overall health of the patient and likelihood of an “Adverse Event,” i.e., hospitalization or severe complications from a clinical incident, fall, etc. High=5×, Very High is 10×. This patient is the only one where the 5×high risk is added on top of their other risk factors.

FIG. 6C shows a patient that is a 55-year-old male with risk scores for overall risk (2.7 low), stroke risk (3.5 low), heart attack risk (8.2 low), and breast cancer risk (22.4 high). The patient depicted in FIG. 6C is 28 days out for MAMMO with a total risk score of 33.0. The risk factor for MAMMO is X3.0 because the patient is a high risk for breast cancer. The goal factor for MAMMO is X10.0 because reducing breast cancer is a very important goal.

As shown in FIGS. 6A-6C, the patient shown in FIG. 6B has a much higher total communication priority because of a very high HTN MISSING BP, even though the patient is not out of compliance by very many days. In this case, the risk factors for HTN MISSING BP drive the high risk score for HTN MISSING BP. On the other hand, the patient shown in FIG. 6C has a higher total communication priority because of a risk factor for high breast cancer risk and because of a high goal factor for MISSING MAMMO.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firework, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the users computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PIA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks, These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

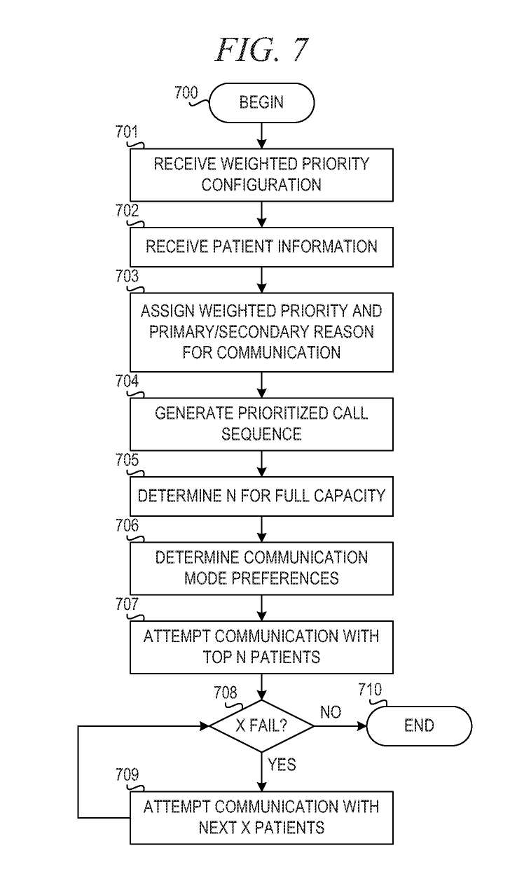

FIG. 7 is a flowchart illustrating operation of a system for patient communication priority by compliance dates, risk scores, and organizational goals in accordance with an illustrative embodiment. Operation begins (block 700), and the system receives a weighted priority configuration (block 701) and receives patient information for patients out of compliance (block 702). The system assigns weighted priority and primary/secondary reasons for communication (block 703). The system generates a prioritized call sequence (block 704).

The system determines N for full capacity of the provider/facility (block 705). The, the system determines the communication mode preferences for the patients who are out of compliance (block 706). The system then attempts communication with top N patients (block 707). The system determines Whether communication fails for any X greater than zero (block 708). If communication fails for X patients, then the system attempts communication with the next X patients (block 709), and operation returns to block 708 to determine if communication fails for any patients. If communication is successful for all patients in block 708, then operation ends (block 710).

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Thus, the illustrative embodiments provide a mechanism that prioritizes patient communication by compliance dates, risk scores, and organizational goals. The illustrative embodiments prioritizes patients with higher risk scores associated with a condition who are further out of compliance over patients with a lower risk score and are closer to being in compliance. The illustrative embodiments also take into account the seriousness of the condition and the goals of the organization to drive more patients to goal for targeted metrics for the organization. Thus, the illustrative embodiments balance high risk patient needs with organizational goals.

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

1. A method, in a data processing system comprising at least one processor and at least one memory, the at least one memory comprising instructions executed by the at least one processor to cause the at least one processor to implement a clinical decision support system, the method comprising:
receiving, by a priority calculation component executing within the clinical decision support system, weighted priority configuration for a health care facility and a set of patients with care gaps;
assigning, by the priority calculation component, a priority value to each given patient in the set of patients based on a risk score of at least one condition for the given patient, a number of days out of compliance for the at least one condition for the given patient, and an organizational goal factor associated with the at least one condition for the health care organization;
generating, by the priority calculation component, a prioritized communication sequence for the set of patients; and
initiating, by a communication engine executing within the clinical decision support system, communication with patients within the set of patients based on the prioritized communication sequence.

2. The method of claim 1, wherein the weighted priority configuration comprises compliance priorities by reason, risk priority factors for a set of complications, and organizational goal factors for the set of complications and wherein the set of patients have associated patient information comprising the number of days out of compliance and the risk score associated with the at least one condition.

3. The method of claim 2, wherein assigning the priority value comprises:
determining a risk priority score for the at least one complication based on the risk score and the number of days out of compliance; and
applying a risk priority factor and an organizational goal factor associated with the at least one complication to the risk priority score.

4. The method of claim 3, wherein applying the risk priority factor comprises applying the risk priority factor dependent upon a level of risk of the complication for the given patient.

5. The method of claim 3, wherein assigning the priority value comprises determining the priority value based on all risk priority scores for the given patient.

6. The method of claim 1, further comprising:
identifying a first subset of patients having highest priority values within the set of patients;
responsive to communication with a number of patients not being successful, identifying a second subset of patients and initiating communication with the second subset of patients until the health care organization is at full capacity.

7. The method of claim 1, wherein the weighted priority configuration comprises a set of days out of compliance factors for a plurality of tiers of days out of compliance.

8. The method of claim 7, wherein each of the plurality of tiers of days out of compliance has a corresponding weight such that a tier with a higher number of days out of compliance has a higher weight than a tier with a lower number of days out of compliance.

9-20. (canceled)
